(19) United States
(12) Patent Application Publication
(10) Pub. No.: US 2025/0366784 A1
(43) Pub. Date: Dec. 4, 2025

(54) HAND TREMOR TREATMENT DEVICE

(71) Applicant: Imam Abdulrahman Bin Faisal University, Dammam (SA)

(72) Inventors: Mariyah Shafiq AL-MUBARAK, Dammam (SA); Hala EL-WAKEEL, Dammam (SA)

(73) Assignee: Imam Abdulrahman Bin Faisal University, Dammam (SA)

(21) Appl. No.: 19/067,511

(22) Filed: Feb. 28, 2025

Related U.S. Application Data
(60) Provisional application No. 63/652,892, filed on May 29, 2024.

(51) Int. Cl. A61B 5/00; A61B 5/11; A61H 9/00; A61H 23/02

(52) U.S. Cl. CPC A61B 5/4836; A61B 5/1101; A61B 5/1126; A61B 5/6824; A61B 5/7475; A61H 9/0078; A61H 23/0254; A61B 2560/0462; A61B 2562/0219; A61H 2201/0153; A61H 2201/1215; A61H 2201/123; A61H 2201/1238; A61H 2201/1638; A61H 2201/5023; A61H 2201/5084; A61H 2201/5097

(57) ABSTRACT

A hand tremor treatment device that includes a ventral pad including a ventral vibration-inducing device and a ventral compression-inducing device, a dorsal pad including a dorsal vibration-inducing device and a dorsal compression-inducing device, a lateral strap, a medial strap, and a controller. The device is configured to be worn on the wrist and is configured to detect a hand tremor onset and in response induce compression via at least one selected from the ventral compression-inducing device and the dorsal compression-inducing device and to induce vibration via at least one selected from the ventral vibration-inducing device and the dorsal vibration-inducing device.

### CROSS-REFERENCE TO RELATED APPLICATIONS

The present application claims priority to U.S. Provisional Application No. 63/652,892 filed May 29, 2024, which is incorporated by reference in its entirety for all purposes.

### BACKGROUND OF THE INVENTION

### Field of the Invention

The present disclosure relates to a hand tremor treatment device and a method for treating hand tremors.

### Discussion of the Background

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

A tremor is an involuntary, muscle contraction leading to shaking, rhythmic, repetitive, or cyclic movement in one or more parts of the body. The muscle contraction often follows a rhythmic pattern. A common site of tremors is the hand or wrist area, but tremors can also occur in the arms, legs, head, torso, feet, and other body parts as well. A tremor may be intermittent or can be constant or permanent. The effects of tremors can be incredibly disruptive to patients suffering them. Tremors can impair normal bodily functions, rendering everyday tasks difficult or impossible. Tremors can also impair a patient's mental health, being a source of frustration or embarrassment. Examples of types of tremors include essential tremor, restless leg syndrome (RLS), Parkinson's tremor, dystonic tremor, cerebellar tremor, resting tremor, action tremor, psychogenic tremor, enhanced physiologic tremor, or orthostatic tremor. Tremors impact many aspects of the patient's daily living and interfere with many physical activities at home and in the workplace. Additionally, among patients suffering from tremor, their psychological strain may be significantly more affected

Typically, tremors may be treated with medications. Examples of medications currently used to treat types of tremor include anti-seizure medications, including topiramate or gabapentin, beta blockers, such as propranolol, atenolol, metoprolol, nadolol, and sotalol, benzodiazepine tranquilizers, such as alprazolam or clonazepam, Parkinson's disease medications, such as levodopa or carbidopa, and in some cases, medications such as botulinum toxin (BTX). However, medications may have certain side effects that are undesirable for the particular sufferer of tremor.

In some cases, particularly for severe or debilitating tremors or tremor disorders that do not respond to medication, surgery may be performed to treat the tremor. Though improvement can be seen after these procedures, the procedures may also be the cause of other debilitating effects, such as speech, movement, or balance issues.

High frequency transcutaneous electrical nerve stimulation (TENS) has been used in the treatment of various movement disorders, including myoclonic dystonia and ET. While the exact mechanism of TENS remains unclear, it is typically attributed to an ability to affect transmission of sensory information from the periphery to the central nervous system. Many studies suggest that treatment with TENS in patients who have tremors was associated with improved muscle strength and tremor reduction. However, this treatment option is frequently associated with muscle fatigue, numbness, burning sensations, and even nerve damage with prolonged use.

Tremor suppression orthoses for the upper limbs, wrist, and elbow joints have been studied. These devices are typically classified into active, semi-active, or passive. Active orthoses work by generating an active force that counteracts the involuntary motions while supporting the voluntary motions in patients with tremors. In contrast, semi-active and passive orthoses use energy dissipation or absorption to suppress involuntary movements. However, such devices may not be effective for all types of tremors and can be limited based on the orientation of the device compared with the direction of the tremor motion. Further, such devices may require large, heavy masses to effectively dampen motion.

As such, there remains a need to develop tremor treatment devices that can overcome these drawbacks.

### SUMMARY OF THE INVENTION

The present disclosure relates to a hand tremor treatment device. In some embodiments, the hand tremor treatment device comprises a ventral pad configured to be positioned on a ventral surface of a wrist of a subject and including a ventral vibration-inducing device and a ventral compression-inducing device, a dorsal pad configured to be positioned on a dorsal surface of a wrist of the subject and including a dorsal vibration-inducing device and a dorsal compression-inducing device, a lateral strap connecting the ventral pad and the dorsal pad and configured to pass over a lateral surface of a wrist of the subject, a medial strap connecting the ventral pad and the dorsal pad and configured to pass over a medial surface of a wrist of the subject, and a controller configured to detect a hand tremor onset and in response to detecting the hand tremor onset induce compression via at least one selected from the ventral compression-inducing device and the dorsal compression-inducing device and to induce vibration via at least one selected from the ventral vibration-inducing device and the dorsal vibration-inducing device.

In some embodiments, the lateral strap and the medial strap are flexible.

In some embodiments, at least one selected from the group consisting of the lateral strap and the medial strap is adjustable to securely conform to a wrist of a subject.

In some embodiments, the ventral compression-inducing device and the dorsal compression-inducing device each include an inflatable airbag.

In some embodiments, the inflatable airbag includes a pump.

In some embodiments, the pump is configured to inflate and deflate the airbag in response to an airbag signal from the controller.

In some embodiments, the ventral vibration-inducing device and the dorsal vibration-inducing device are each selected from the group consisting of a rotary vibration motor and a piezoelectric linear vibration motor.

In some embodiments, the hand tremor treatment device comprises at least one vibration-inducing device configured to produce vibration in a first frequency range and at least one vibration-inducing device configured to produce vibration in a second frequency range.

In some embodiments, the first frequency range is 0.1 Hz to 1 kHz.

In some embodiments, the second frequency range is 10 kHz to 2.5 MHz.

In some embodiments, the hand tremor treatment device further comprises an accelerometer, wherein the controller is configured to detect hand tremor onset using the accelerometer.

In some embodiments, the hand tremor treatment device further comprises an angular velocity sensor, wherein the controller is configured to detect hand tremor onset using the angular velocity sensor.

In some embodiments, the ventral pad further comprises a user input device.

The present disclosure also relates to a hand tremor treatment system. In some embodiments, the hand tremor treatment system comprises a first hand tremor treatment device, comprising a first ventral pad configured to be positioned on a ventral surface of a wrist of a subject and including a first ventral compression-inducing device and a first ventral vibration-inducing device, a first dorsal pad configured to be positioned on a dorsal surface of a wrist of the subject and including a dorsal compression-inducing device and a first dorsal vibration-inducing device, a first lateral strap connecting the first ventral pad and the first dorsal pad and configured to pass over a lateral surface of a wrist of the subject, a first medial strap connecting the first ventral pad and the first dorsal pad and configured to pass over a medial surface of a wrist of the subject, a first wireless communication device, and a controller, and a second hand tremor treatment device, comprising a second ventral pad configured to be positioned on a ventral surface of a wrist of a subject and including a ventral compression-inducing device and a second ventral vibration-inducing device, a second dorsal pad configured to be positioned on a dorsal surface of a wrist of the subject and including a dorsal compression-inducing device and a second dorsal vibration-inducing device, a second lateral strap connecting the second ventral pad and the second dorsal pad and configured to pass over a lateral surface of a wrist of the subject, a second medial strap connecting the second ventral pad and the second dorsal pad and configured to pass over a medial surface of a wrist of the subject, and a second wireless communication device, wherein the controller is configured to detect a hand tremor onset and in response to detecting the hand tremor onset induce compression via at least one selected from the first ventral compression-inducing device, the first dorsal compression-inducing device, the second ventral compression-inducing device, and the second dorsal compression-inducing device and to induce vibration via at least one selected from the first ventral vibration-inducing device, the first dorsal vibration-inducing device, the second ventral vibration-inducing device and the second dorsal vibration-inducing device, and the first wireless communication device is configured to receive a signal from the controller and transmit the signal to the second wireless communication device.

In some embodiments, the first ventral pad further comprises a user input device.

The present disclosure also relates to a method of treating a hand tremor in a subject, the method comprising detecting a hand tremor onset using the hand tremor treatment device and inducing compression via at least one selected from the ventral compression-inducing device and the dorsal compression-inducing device and to induce vibration via at least one selected from the ventral vibration-inducing device and the dorsal vibration-inducing device.

In some embodiments, the method further comprises determining a hand tremor movement frequency, and inducing vibration based on the hand tremor frequency.

In some embodiments, the controller comprises an angular velocity sensor and the controller is configured to detect hand tremor onset using the angular velocity sensor.

In some embodiments, the controller comprises an accelerometer and the controller is configured to detect hand tremor onset using the accelerometer.

In some embodiments, the method further comprises determining a hand tremor movement intensity, and inducing vibration and compression based on the hand tremor intensity.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIGS. 1A-1B show exemplary hand tremor treatment devices that may form a hand tremor treatment system, according to certain embodiments, with FIG. 1A showing a first device and FIG. 1B showing a second device.

FIGS. 2A-2B show exemplary hand tremor treatment devices that may form a hand tremor treatment system, according to certain embodiments, with FIG. 2A showing a first device and FIG. 2B showing a second device.

FIG. 3 shows an exemplary hand tremor treatment device according to certain embodiments.

FIG. 4 shows a schematic drawing of an exemplary hand tremor treatment device according to certain embodiments.

FIG. 5 is an illustration of a non-limiting example of details of computing hardware used in a computing system corresponding to a controller of the additive manufacturing system, according to certain embodiments;

FIG. 6 is an exemplary schematic diagram of a data processing system used within the computing system, according to certain embodiments;

FIG. 7 is an exemplary schematic diagram of a processor used with the computing system, according to certain embodiments; and

FIG. 8 is an illustration of a non-limiting example of distributed components which may share processing with the controller, according to certain embodiments.

### DETAILED DESCRIPTION OF THE INVENTION

In the following description, it is understood that other embodiments may be utilized and structural and operational changes may be made without departure from the scope of the present embodiments disclosed herein.

### Definitions

As used herein the words “a” and “an” and the like carry the meaning of “one or more.”

As used herein, the terms “optional” or “optionally” means that the subsequently described event(s) can or cannot occur or the subsequently described component(s) may or may not be present (e.g., 0 wt. %).

According to a first aspect, the present disclosure relates to a hand tremor treatment device. In some embodiments, the hand tremor treatment device comprises a ventral pad and a dorsal pad. In some embodiments, the hand tremor treatment device is configured to be worn on a wrist of a subject. In some embodiments, the ventral pad is configured to be positioned on a ventral surface of a wrist of a subject. In some embodiments, the dorsal pad is configured to be positioned on a dorsal surface of a wrist of a subject.

In some embodiments, the ventral pad includes a ventral vibration-inducing device and a ventral compression-inducing device. In some embodiments, the dorsal pad includes a dorsal vibration-inducing device and a dorsal compression-inducing device.

In general, the ventral vibration-inducing device and the dorsal vibration-inducing device can each be any suitable vibration-inducing device known to one of ordinary skill in the art. Examples of vibration-inducing devices include, but are not limited to a piezoelectric linear vibration motor (also referred to as a piezoelectric actuator), a rotary vibration motor, a linear vibration motor (also referred to as a linear resonant actuator), and a solenoid vibration motor.

A rotary vibration motor can include an off-center or off-balance mass connected to a central rotating portion. Such a rotary vibration motor can be referred to as a “rotating eccentric mass vibration motor”. Linear Resonant Actuators (LRAs) are commonly found in wearables and smartphones. LRA motors can include a magnet attached to a spring, surrounded by an electromagnetic coil and housed in a casing. The coil is used to drive the motor by moving the mass back and forth within the housing, creating the vibrations. Piezoelectric actuators function by applying a voltage to piezoelectric material, which causes it to change shape and produce vibration. These actuators can produce highly detailed haptic feedback, making them advantageous for applications where precision is key, such as in medical devices. Linear Magnetic Ram (LMR) functions using solid-state magnetic suspension technology. LMR vibration motors produce vibration by driving a suspended mass through a magnetic field. This movement is controlled by an electric current, which can be adjusted to change the position, speed, and force of the mass. In some embodiments, the ventral vibration-inducing device and the dorsal vibration-inducing device are each selected from the group consisting of a rotary vibration motor and a piezoelectric linear vibration motor.

In some embodiments, the ventral vibration-inducing device and the dorsal vibration-inducing device are the same type of vibration-inducing device. For example, both the ventral vibration-inducing device and the dorsal vibration-inducing device can be piezoelectric linear vibration motors. In some embodiments, the ventral vibration-inducing device and the dorsal vibration-inducing device are different types of vibration-inducing device. For example, the ventral vibration-inducing device can be a linear magnetic ram vibration motor and the dorsal vibration-inducing device can be a piezoelectric linear vibration motors.

In some embodiments, the hand tremor treatment device is configured to produce vibration in a first frequency range and vibration in a second frequency range. In some embodiments, the hand tremor treatment device comprises at least one vibration-inducing device configured to produce vibration in a first frequency range and at least one vibration-inducing device configured to produce vibration in a second frequency range. In some embodiments, a single vibration-inducing device is capable of producing both the vibration in a first frequency range and the vibration in a second frequency range. For example, a single vibration-inducing device may be configured to vibrate at multiple frequencies, for example a fundamental frequency (or first harmonic) and a second harmonic.

In some embodiments, the first frequency range is 0.1 Hz to 1 kHz. For example, vibration in the first frequency range can be 0.5 Hz, 1 Hz, 1.5 Hz, 2 Hz, 2.5 Hz, 3 Hz, 3.5 Hz, 4 Hz, 4.5 Hz, 5 Hz, 6 Hz, 7 Hz, 8 Hz, 9 Hz, 10 Hz, 15 Hz, 20 Hz, 25 Hz, 30 Hz, 35 Hz, 40 Hz, 45 Hz, 50 Hz, 55 Hz, 60 Hz, 65 Hz, 70 Hz, 75 Hz, 80 Hz, 85 Hz, 90 Hz, 100 Hz, 125 Hz, 150 Hz, 175 Hz, 200 Hz, 225 Hz, 250 Hz, 275 Hz, 300 Hz, 350 Hz, 400 Hz, 450 Hz, 500 Hz, 550 Hz, 600 Hz, 650 Hz, 700 Hz, 750 Hz, 800 Hz, 850 Hz, 900 Hz, 950 Hz, or the like. Vibrations in this frequency range may be advantageous for disrupting or dampening the movement of a patient's limb (e.g., arm, wrist, hand, etc.). Such disrupting or dampening can be associated with or caused by a mechanical interaction with the limb.

In some embodiments, the second frequency range is 10 kHz to 2.5 MHz. For example, the vibration in the second frequency range can be 10 kHz, 12.5 kHz, 15 kHz, 17.5 kHz, 20 kHz, 22.5 kHz, 25 kHz, 27.5 kHz, 30 kHz, 35 kHz, 40 kHz, 45 kHz, 50 kHz, 55 kHz, 60 kHz, 65 kHz, 70 kHz, 75 kHz, 80 kHz, 85 kHz, 90 kHz, 95 kHz, 100 kHz, 125 kHz, 150 kHz, 175 kHz, 200 kHz, 225 kHz, 250 kHz, 275 kHz, 300 kHz, 325 kHz, 350 kHz, 375 kHz, 400 kHz, 425 kHz, 450 kHz, 475 kHz, 500 kHz, 525 kHz, 550 kHz, 575 kHz, 600 kHz, 625 kHz, 650 kHz, 675 kHz, 700 kHz, 725 kHz, 750 kHz, 775 kHz, 800 kHz, 825 kHz, 850 kHz, 875 kHz, 900 kHz, 925 kHz, 950 kHz, 975 kHz, 1 MHz, 1.100 MHz, 1.125 MHz, 1.150 MHz, 1.175 MHz, 1.200 MHz, 1.225 MHz, 1.250 MHz, 1.275 MHz, 1.300 MHz, 1.325 MHz, 1.350 MHz, 1.375 MHz, 1.400 MHz, 1.425 MHz, 1.450 MHz, 1.475 MHz, 1.500 MHz, 1.525 MHz, 1.550 MHz, 1.575 MHz, 1.600 MHz, 1.625 MHz, 1.650 MHz, 1.675 MHz, 1.700 MHz, 1.725 MHz, 1.750 MHz, 1.775 MHz, 1.800 MHz, 1.825 MHz, 1.850 MHz, 1.875 MHz, 1.900 MHz, 1.925 MHz, 1.950 MHz, 1.975 MHz, 2.0 MHz, 2.1 MHz, 2.2 MHz, 2.3 MHz, 2.4 MHz, or 2.5 MHz. Vibrations in this frequency range may be advantageous for stimulating nerves, such as the median nerve in the arm.

In general, there is no limit to the number of vibration-inducing devices that can be included in the dorsal pad and/or the ventral pad. For example, the dorsal pad can include a vibration-inducing device configured to operate in the first frequency range and a vibration-inducing devices configured to operate in the second frequency range; the dorsal pad can include a vibration-inducing device configured to operate in the first frequency range while the ventral pad includes a vibration-inducing devices configured to operate in the second frequency range or vice-versa; the dorsal pad and/or ventral pad can each include a single vibration-inducing device configured to operate in both the first frequency range and the second frequency range, or some combination of these.

In general, the ventral compression-inducing device and the dorsal compression-inducing device can each be any suitable compression-inducing device known to one of ordinary skill in the art. Examples of compression-inducing devices include, but are not limited to linear actuators, rotary actuators, pistons, and inflatable airbags/bladders. In general, there is no limit to the number of compression-inducing devices that can be included in the dorsal pad and/or the ventral pad. In some embodiments, the compression-inducing device or devices are configured to only apply compression to a dorsal and/or ventral surface of the wrist. In some embodiments, the compression-inducing device or devices do not apply compression to a medial and/or lateral surface of the wrist. In some embodiments, the compression-inducing device does not totally encompass the wrist, for example, as cuff.

In some embodiments, the dorsal compression-inducing device and the ventral compression-inducing device each include an inflatable airbag. In some embodiments, the inflatable airbag includes a pump. In some embodiments, the pump is configured to inflate and deflate the inflatable airbag. In some embodiments, the pump is configured to inflate and deflate the inflatable airbag in response to an airbag signal from the controller. Such an airbag signal can include an instruction for the pump to initiate an inflation, initiate a deflation, change a compression pressure, maintain a certain compression pressure, re-fill an airbag, or other similar function. In some embodiments, the inflatable airbag includes a pressure sensor. The pressure sensor can be configured to monitor a pressure inside the inflatable airbag. In some embodiments, the pressure sensor can be in communication with the controller and/or pump. The pressure sensor may be advantageous for detecting the compression pressure provided by the inflatable airbag, for ensuring that proper compression pressure is achieved, for detecting leaks, for ensuring that compression is ceased at a proper time, or the like.

In some embodiments, the dorsal compression-inducing device and/or the ventral compression-inducing device includes a vibration transmitting member. The vibration transmitting member can be an extendible structure configured to transmit vibration from one or more vibration-inducing devices to the user's wrist (e.g., the dorsal wrist surface and/or ventral wrist surface) when the inflatable airbag is inflated. When an inflatable airbag is inflated, there may be an air gap between the surface of the wrist in contact with the inflatable air bag and the vibration-inducing device. This air gap may interfere with transmission of the vibration to the subject, lowering the efficacy of the tremor intervention and/or requiring a higher vibration intensity to reach the same efficacy. A vibration transmitting member can be a suitable structure that may be sufficiently rigid to pass the vibrations effectively from the vibration-inducing device to the user's wrist. In some embodiments, the vibration transmitting member may be folded, hinged, or structured like an accordion to allow the vibration transmitting member to extend from a deflated position when the airbag is deflated to an inflated position when the airbag is inflated.

In some embodiments, the airbag includes a wrist contact plate. The wrist contact plate can be a suitable rigid plate configured to contact the user's wrist (e.g., the dorsal wrist surface or ventral wrist surface). In some embodiments, the wrist contact plate can be disposed such that inflation of the airbag pushes the wrist contact plate away from other portions of the device toward the user's wrist. For example, the wrist contact plate can be position on an exterior of the airbag such that the airbag separates the wrist contact plate from other portions of the device. In some embodiments, the vibrating transmitting member is coupled to the wrist contact plate. In some embodiments, the wrist contact plate is substantially flat. In some embodiments, the wrist contact plate is concave. Such a concave shape may be configured to conform to a user's wrist.

In some embodiments, the device comprises a display. In some embodiments, the display is disposed at an exterior of the dorsal pad. Such a location may mimic the location of the face of a watch worn on the user's wrist. In general, the display can be any suitable type of display. In general, the display can display any suitable information that may be advantageous for a user to have displayed on their wrist. For example, the display can be configured to display any suitable information that may be displayed by a watch or smartwatch. In some embodiments, the display displays information that may be transmitted to the display from a portable electronic device (e.g., smartphone). Such a transmission may be achieved via the wireless communication device described below.

In some embodiments, the device comprises a user input device. The user input device can be any suitable such device known to one of ordinary skill in the art. The user input device can include a touchscreen. For example, the electronic user input device can be a keypad and/or touchscreen. The keypad may be a physical keypad and can include any suitable buttons, such as dedicated buttons or context-dependent buttons. The user input device is configured to receive user input and provide instructions to the controller based on the user inputs. Such user inputs can be, include, or correspond to any action related to a function of the device and/or treatment of a tremor, such as providing input relating to the intensity of compression and/or vibration, a tremor sensitivity, or some other function not related to the treatment of a tremor, such as setting a clock or acknowledging a notification. For example, the user input device can be used to receive user inputs that cause the device to initiate compression and/or vibration if the user wants to manually activate treatment of tremor.

In some embodiments, the device includes a wireless communication device. The wireless communication device can enable or facilitate transmission of electronic information between various components of the device, between devices of the hand tremor treatment system, and/or other devices, such as a user's portable electronic device. For example, the wireless communication device can be used to transmit limb data from a device worn on a patient's wrist that does not include a controller but is still equipped to detect and provide treatment and/or intervention for a tremor (e.g., a secondary device or non-controller device) to a device that includes a controller (e.g., a user's portable electronic device, a primary or controller-equipped device). This may allow the non-controller device to detect and provide intervention for a tremor in the limb on which it is worn as part of a system that only requires a single controller. In general, the wireless communication module can use any protocol or method for transmitting electronic information, such as Wifi, Bluetooth®, AirPlay®, EDGE, wireless cellular systems such as 3G, 4G and 5G, and the like.

In some embodiments, the device can include a power source. Examples of power sources include, but are not limited to a battery, a capacitor, a piezoelectric device, and other forms of energy harvesting devices. The power source may be useful for supplying electrical power to the components of the device, such as sensors, the vibration-inducing device, the compression-inducing device, the controller, the wireless communication module, and/or other components.

The power source may be a rechargeable battery or a number of rechargeable batteries. The device may include appropriate hardware for charging the rechargeable battery/batteries. For example, the device may include a charging port configured to accept a charging cable for supplying electrical power to the rechargeable battery/batteries. The cable may be any such cable such as such as USB, mini-USB or the like, a wall outlet, an automobile 12V power, a renewable power source or the like. In another example, the device may include a wireless charging receiver interface. Such a wireless charging receiver can be configured to connect to or interface with an appropriate wireless charging supplier interface. Such connecting or interfacing may be accomplished with magnets, reusable adhesive, mechanical interference fit clamps, or any other method. In another example, the rechargeable battery/batteries (and/or other power source) may be configured to be removed and replaced such that the power source can be swapped by the subject (user) as needed.

In some embodiments, the device includes a notification device. In general, the notification device can be any suitable device capable of alerting, notifying, or otherwise getting a subject's (user's) attention. In general, the notification device can be or include a device for generating an audible notification (e.g., a noise), a tactile notification such as a vibration, rumble, shake, or the like, a visual notification such as a light, flash, blink, color change, or the like, a visual notification such as a light, flash, or display on a screen, or a combination of these. In general, the notification device can be or include any suitable hardware for generating the aforementioned notification(s). For example, the notification device can include a vibration-inducing device. Such a vibration-inducing device can be the same as a vibration-inducing device configured to tremor treatment as described above or can be a separate vibration-inducing device, such as one dedicate to use in notification. The notification device, for example, can be or include a light such as an LED. The LED can blink, flash, change color, or otherwise change some feature of its operation to provide the notification. The notification device, for example, can be or include a speaker. The speaker can, for example, play a specific sound to provide the notification. The speaker can, for example, play a pre-recorded or procedurally generated voice to provide the notification.

In some embodiments, the device includes a cover. In some embodiments, the cover can be disposed upon the dorsal pad, the ventral pad, or both. In some embodiments, the device includes a dorsal cover configured to be placed over the dorsal pad. In some embodiments, the device includes a ventral cover configure to be placed over the ventral pad. In general, the cover(s) can be formed from any suitable material described above. In some embodiments, the cover or covers are formed from a rigid material, such as metal or a rigid polymer. The rigid material may serve to or be advantageous for providing protection to the device or portion thereof disposed under the cover. In some embodiments, the rigid material may be further covered by another material, such as a textile, silicone, flexible polymer, or the like. This other material may be advantageous for protecting a subject (user) or for increasing subject (user) comfort.

In some embodiments, the ventral pad and dorsal pad can be connected by one or more connecting members. The connecting member can be configured to pass over a lateral surface of a wrist of the subject and/or a medial surface of a wrist of the subject. In some embodiments, the connecting member can be rigid. That is, the connecting member maintains approximately the same shape. A rigid connecting member can be configured to flex or to hinge open to allow a user to put on or take off the device. In some embodiments, the connecting member can be flexible. That is, the connecting member does not maintain approximately the same shape.

In embodiments with one connecting member, the connecting member can be configured to encompass a portion but not an entirety of a wrist of the subject. Alternatively, in embodiments with one connecting member, the connecting member can be configured to encompass an entirety of a wrist of the subject. For example, the connecting member can be a single strap that is configured to pass over both the lateral and medial surfaces of the wrist and connect the ventral pad and the dorsal pad. Such a single strap can also be configured to pass over a dorsal and/or ventral surface of the wrist. For example, the single strap can pass over the dorsal and/or ventral pad such that the dorsal and/or ventral pad remains in contact with the appropriate wrist surface. In some embodiments, the single strap can pass around a side of a dorsal and/or ventral pad.

In some embodiments, the device includes a pair of connecting members. In some embodiments, the pair of connecting members includes a lateral connecting member that connects the ventral pad and the dorsal pad and is configured to pass over a lateral surface of a wrist of the subject and a medial connecting member that connects the ventral pad and the dorsal pad and is configured to pass over a medial surface of a wrist of the subject. In some embodiments, the lateral connecting member is flexible. In some embodiments, the medial connecting member is flexible. In some embodiments, both the lateral connecting member and the medial connecting member are flexible. In some embodiments, at least one selected from the group consisting of the lateral connecting member and the medial connecting member is adjustable to securely conform to a wrist of a subject. In some embodiments, the lateral connecting member is a strap. In some embodiments, the medial connecting member is as strap.

In general, a strap used as a connecting member can be secured using any suitable fastening or securing mechanism. Examples of such mechanisms include, a clip, a clasp, a buckle, a cam lock, a hook-and-loop fastener, a hook-and-hook fastener, and/or a slidingly engaging fastener. In some embodiments, the strap can be adjustable. That is, a length of the strap can be changed and/or the strap can be secured in a different position to accommodate limbs of different sizes. For example, the strap can include a plurality of fastening structures (e.g., snaps, loops, slots, indentations, etc.) arranged at different positions along a length of the strap. The plurality of fastening structures can allow the strap to be securely fastened around wrists of various sizes.

In some embodiments, the connecting member or strap can be formed from a stiff but flexible material that is capable of flexing to allow the user to place the device on their wrist. In some embodiments, the connecting member or strap is formed from a metal or stiff polymer band coated with a body-safe material. A body-safe material is any material which is generally recognized as safe for contact with the skin. A body-safe material may be hypoallergenic. Examples of materials suitable for forming the connecting member or strap include, but are not limited to foams, rubbers, polymers, elastomers, textiles such as cloths, leathers, metals, and the like. Specific examples of suitable materials include, but are not limited to polyisoprene, polybutadiene, chloroprene, neoprene, butyl rubber, styrene-butadiene rubber, nitrile rubber, ethylene polypropylene rubber, epichlorohydrin rubber, acrylic rubber, silicone rubber, fluorosilicone rubber, fluoroelastomers such as FKM and FEPM, perfluoroelastomers such as FFKM, polyether block amides, ethylene-vinyl acetate, silicone elastomers such as polydimethylsiloxane (PDMS) or other linear polysiloxanes and network polysiloxanes such as those present in silicone resins, EPDM rubber, polyvinylchloride foam, polyurethane foam, latex foam, and combinations of these.

In some embodiments, the hand tremor treatment device can include a motion sensor. In general, the motion sensor can be any suitable sensor capable of detecting and/or determining the properties of motion (e.g., frequency, force, acceleration, direction, etc.). Examples of such sensors include, but are not limited to flow sensors, accelerometers, gyroscopes, inertial sensors, magnetic sensors, angular velocity sensors, and combinations thereof. In some embodiments, the motion sensor is configured to measure at least one selected from the group consisting of a three-dimensional limb motion velocity, a three-dimensional limb motion acceleration, and a limb motion frequency. The measurements obtained by the limb sensor can be referred to as “limb data”, “limb sensor data”, or other similar term. In some embodiments, the motion sensor includes an accelerometer and a gyroscopic sensor. In such an embodiment, the gyroscopic sensor can provide data relating to an orientation of the limb and/or a direction of motion and the accelerometer can provide data relating to the motion of the limb. These two types of data relating to the limb can be combined or integrated to provide an inertial measurement unit. In some embodiments, the inertial measurement unit also includes a magnetometer. The magnetometer can be configured to operate based on the Earth's natural magnetic field or based on a magnetic field from a magnet placed in another component of the system. The magnetometer can provide data relating to an absolute orientation of the limb sensor relative to some external reference source, such as the Earth's natural magnetic field or magnet placed in another component of the system. Preferably, the inertial measurement unit includes a three-axis accelerometer. The three-axis accelerometer can be formed from three individual one-axis accelerometers arranged so as to provide three orthogonal axes of acceleration measurement. In some embodiments, the hand tremor treatment device uses the inertial measurement unit to detect a limb motion (e.g., a hand motion, wrist motion, arm motion, etc.). That is, the inertial measurement unit is configured to track changes in the limb position and orientation to provide the measured limb motion. In some embodiments, the limb sensor is configured to provide a measurable signal to the controller. In some embodiments, the measurable signal is useful for detecting a hand tremor.

In some embodiments, the hand tremor treatment device includes a controller. In some embodiments, the controller is configured to detect a hand tremor onset and in response to detecting the hand tremor onset initiate a tremor intervention (e.g., compression and/or vibration intended to treat or reduce the intensity of the tremor). The initiate of the intervention can include inducing compression via at least one selected from the ventral compression-inducing device and the dorsal compression-inducing device and inducing vibration via at least one selected from the ventral vibration-inducing device and the dorsal vibration-inducing device. In some embodiments, the controller is configured to control the properties of the compression and/or the vibration. For example, the controller can adjust an intensity of the compression and/or the vibration to produce effective treatment of the tremor. Such intensity of the intervention can be based on the detected intensity of the tremor. Such adjustment can optionally take into account other factors such as user comfort, tremor type, tremor intensity, time of day, and user settings.

In some embodiments, the controller is configured to initiate a tremor treatment (e.g., to imitate compression and/or vibration) automatically in response to detecting a tremor. A tremor can be detected based on limb data collected by the sensors described above. In some embodiments, automatic tremor detection may be performed using a machine learning model to generate one or more output values based on one or more input variables (e.g., limb data variables). A machine learning model may be trained before it is used to make an inference from new input data. Training a machine learning model may involve, for example, determining parameters of the machine learning model, such as values of weights associated with one or more nodes of a neural network model. In some embodiments, a machine learning model may be trained in a supervised manner using a training data set that includes labeled training data. The labeled training data may include inputs and corresponding annotated outputs that the machine learning model may use to approximate using learned weight values. In some other embodiments, a machine learning model may be trained in an unsupervised manner in which parameters of the machine learning model may be determined without using labeled training data.

In some embodiments, the parameter(s) of a machine learning model may be trained via loss minimization by feeding a training dataset to the machine learning model. For example, training a machine learning model may include optimization of parameters (e.g., weights or biases) of the machine learning model using techniques such as gradient descent and backpropagation techniques. A validation dataset may also be allocated (e.g., about 20%) from the training dataset to validate a trained machine learning model before deploying the machine learning model.

As used herein, a “classifier” may refer to a type of machine learning model that is trained to categorize inputs into one or more classes of a set of classes. Inputting data into a trained classifier may result in an output that categorizes the input data into a tremor in need of intervention and a movement that does not need intervention. Thus, a properly trained classifier may provide an estimation of a mapping between input variables and discrete (as opposed to continuous) output variables. A classifier may be trained, for example, through a supervised fashion (including optimization and backpropagation), where the training data may comprise known and labeled information. In some cases, the trained classifier may use a regression technique such as logistic regression, which uses a logistic function to model a variable (such as motion acceleration, direction, frequency, or the like) that may have multiple possible discrete outcomes, e.g., a binary or dichotomous outcome such as “tremor in need of intervention” or “not a tremor in need of intervention.” Logistic regression can map the predicted values to probabilities using, for example, a sigmoid function. A sigmoid function can map values between one end to another (e.g., 0 to 1), where one end may correspond to a meal and the other end may correspond to not a meal. In some implementations, one or more threshold values may be needed for the classifier to map the input data to one of the discrete outcomes. For example, if the probability is determined to be above a 0.2 threshold needed to be classified as a “0” but below a 0.8 threshold needed to be classified as a “1,” then the machine learning model may consider the determination invalid or null rather than one of the binary outcomes.

In some cases, the trained classifier may use a neural network such as a long short-term memory (LSTM) network, which is a type of recurrent neural network (RNN) capable of learning context and order. A RNN may perform the same task (looping back) for each successive element of a sequence, e.g., time series data, and the output may depend on the previous calculation. Each element may be associated with a corresponding layer of the RNN. The RNN may store an internal state, akin to a memory, which is determined based on the previously performed task at a previous time step and the current input at a current time step. Unlike a typical RNN, a LSTM may pass additional information (sometimes known as “cell memory”) from one time step to another, which helps capture long-term dependencies through multiple layers, carrying information between points in time and passing new information between states, which is more difficult to do with typical RNNs. That is, LSTMs can better remember previous information and use it for processing the current input. LSTMs can also discard irrelevant information. The output may be based on a sigmoid function (e.g., SoftMax), making LSTMs applicable to binary determinations such as “tremor in need of intervention” or “not a tremor in need of intervention.”

In some embodiments, the controller may be further configured to evaluate the different data provided by one or more sensors to further determine parameters of the motion. Such parameters may be useful in determining the initiation, duration, intensity, or other parameter of the intervention (e.g., the vibration and/or compression) need of the subject. For example, the controller can be configured to determine parameters such as estimates of an activity the user is performing. For example, the controller may be configured to determine based on the data received from the various sensors that the subject (user) is participating in a voluntary activity and is therefore not experiencing a tremor in need of treatment. The controller may be configured to determine based on the data received from the various sensors a vibration intensity and/or compression pressure estimated to be needed to affect treatment of the tremor via the intervention (e.g., the vibration and/or compression). Such estimated intensity needs may be based off previous interventions and/or previous detected tremors experience by the subject.

The parameters of the tremor may be determined using machine learning techniques. In an example, machine learning classifiers may be trained to classify signals received from various sensors (e.g., accelerometer and/or gyroscope signals) to determine the parameters of the tremor such as type, expected duration, expected intensity, expected needed intensity for intervention, etc. Detected tremors and the parameters thereof can be stored in a tremor history. The tremor history may be useful for further training of the machine learning model. For example, the tremor history may be useful in estimating the expected intensity and/or expected duration based on the tremors experienced by the specific user. The parameters of interventions (e.g., intervention duration, vibration intensity, compression pressure, etc.) can be stored in an intervention history. The intervention history may be useful for further training of the machine learning model. For example, the intervention history may be useful in estimating the expected needed vibration and/or compression intensity and/or expected needed intervention duration based on previous interventions.

In some embodiments, the controller may initiate an intervention as described above without the aid of non-sensor devices (i.e. the controller alone receives various data from the various sensors, detects the tremor, and provides an appropriate instruction to the compression-inducing device(s) and/or vibration inducing device(s) to initiate an intervention). In some embodiments, the controller provides an instruction based on the detected limb data. In some embodiments, the controller provides an instruction based on a tremor history, an intervention history, and/or a user setting. Such a tremor history, an intervention history, and/or a user setting may be stored in a suitable memory location, such as in a memory included in the device, a portable electronic device such as a smartphone connected to the device, or another device. In some embodiments, the controller may automatically modify the intervention or a parameter thereof (e.g., duration and/or intensity).

The components of the device described above are merely provided as examples. Persons skilled in the art will recognize various implementations of the wearable device and the components of such implementations. All such implementations and components are contemplated to be within the scope of the present disclosure.

In some embodiments, the hand tremor treatment device can be part of a hand tremor treatment system. In some embodiments, the hand tremor treatment system includes a portable electronic device.

In general, the portable electronic device can be any suitable such device, such as a smartphone, tablet, computer, smartwatch, or similar such device capable of receiving a wireless transmission from the hand tremor treatment device or a component thereof. Such a transmission can be provided by a wireless connection via a suitable wireless communication protocol (e.g., Wi-Fi, Bluetooth, etc.). In general, the portable electronic device can serve multiple functions related to the diabetes management system and/or the use thereof.

For example, a user's smartphone can be wirelessly connected to the hand tremor treatment device or a component thereof such that a notification can be provided to the user via the smartphone. In some embodiments, a notification can be provided even when the user's electronic device (e.g., smartphone) is not currently connected to the hand tremor treatment device or a component thereof. For example, a connection between the smartphone and the hand tremor treatment device or a component thereof can transfer information related to future notifications to the user's smartphone (e.g., low battery warnings). Pre-loaded notifications can be configured to be displayed by the smartphone under appropriate conditions, such as at a certain time.

In some embodiments, the hand tremor treatment device or a component thereof is controlled by a suitable application or piece of software configured to run on the portable electronic device. For example, the portable electronic device can be a smartphone and the software can be a smartphone app. Such a piece of software or app can control the transfer of information between the portable electronic device and the hand tremor treatment device or a component thereof. Such a piece of software can provide the user a convenient interface for controlling the hand tremor treatment device or a component thereof using the portable electronic device. For example, a smartphone running a smartphone app can store a machine learning model for tremor detection or a database associated with such a model. The smartphone app can store the user's tremor history and/or intervention history. The tremor history and/or intervention history can be transmitted to hand tremor treatment device or component thereof (e.g., controller), which can update relevant information stored therein.

The user's portable electronic device can serve as a user input device or interface to allow the user to control, adjust, or provide instructions to the hand tremor treatment device or a component thereof. For example, the keyboard or touchscreen of the portable electronic device can be used as a convenient interface for inputting information into the hand tremor treatment device or a component thereof. Such information can be, for example, whether a detected activity is or is not a tremor. For example, the user can manually enter such information to the controller via a touch screen, manually enter such information into their smartphone and transfer to the controller via some wireless communication protocol (e.g., Wi-Fi, Bluetooth, etc.). The user can automatically retrieve the information related to the hand tremor treatment device and/or tremor interventions.

The user's portable electronic device can serve as a system output device or interface to allow the user to view, access, or analyze the parameters or functions of the hand tremor treatment device or a component thereof. For example, the portable electronic device can serve to display or present a notification described above to the user. In some embodiments, the portable electronic device is configured to provide a user notification based on the user behavior associated with the medication delivered by the system. In some embodiments, the portable electronic device can serve to display to the user or allow adjustment of by the user the settings for the tremor interventions (e.g., vibration intensity, compression pressure, etc.).

The hand tremor treatment device can also be configured to, for example, initiate a tremor intervention based on a user input. For example, a user can provide an instruction to the hand tremor treatment device or controller to initiate a tremor intervention. This may be referred to as “manual” or “user-initiated” intervention.

In some embodiments, the hand tremor treatment system comprises a first hand tremor treatment device and a second hand tremor treatment device. Such a system can be configured such that the first hand tremor treatment device is configured to be worn on one wrist and the second hand tremor treatment device be worn on the other wrist. In some embodiments, one of the hand tremor treatment devices can be configured to be a primary device. Such a primary device can include the controller and/or display, user input device, etc. The other hand tremor treatment device can be a secondary device configured to be operated by signals transmitted from the primary device. This way, only a single controller is necessary to operate both hand tremor treatment devices.

The present disclosure also relates to a method of treating a hand tremor in a subject, the method comprising detecting a hand tremor onset using the hand tremor treatment device and inducing compression via at least one selected from the ventral compression-inducing device and the dorsal compression-inducing device and to induce vibration via at least one selected from the ventral vibration-inducing device and the dorsal vibration-inducing device. In general, the detecting can be performed as described above. In some embodiments, the method further comprises determining a hand tremor movement frequency, and inducing vibration based on the hand tremor frequency.

In some embodiments, the controller comprises an angular velocity sensor and the controller is configured to detect hand tremor onset using the angular velocity sensor. In some embodiments, the controller comprises an accelerometer and the controller is configured to detect hand tremor onset using the accelerometer. In some embodiments, the method further comprises determining a hand tremor movement intensity, and inducing vibration and compression based on the hand tremor intensity.

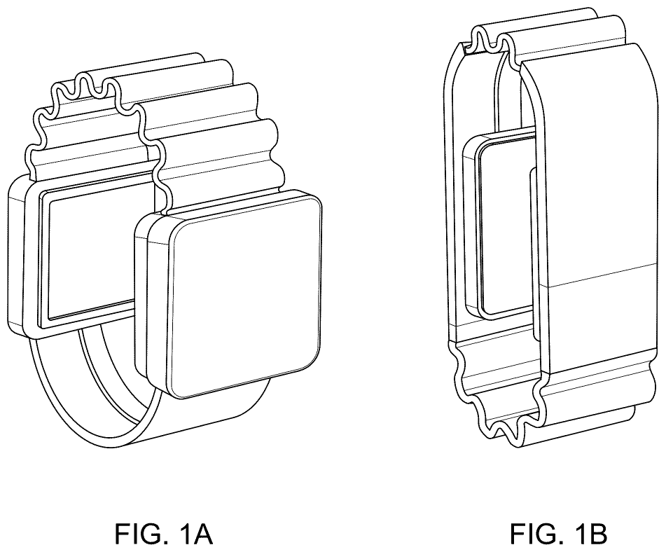

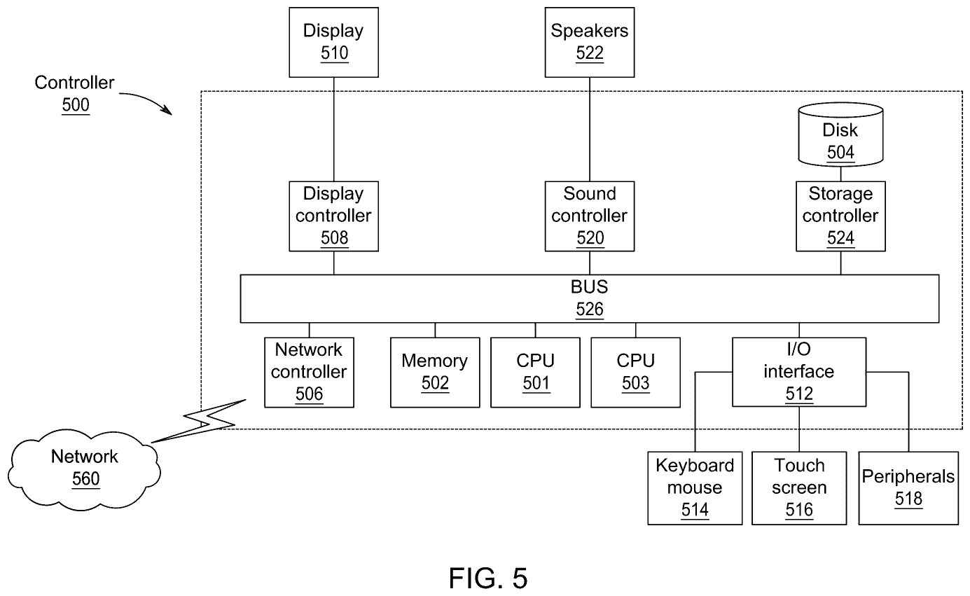

Further details of the hardware description of the computing environment according to exemplary embodiments is described with reference to FIG. 5. In FIG. 5, a controller 500 is described is representative of the controller 120 of FIG. 1A, in which the controller is a computing device which includes a CPU 501 which performs the processes described above/below. In some embodiments, the process data and may instructions be stored in memory 502. In some embodiments, these processes and instructions may alternatively or additionally be stored on a storage medium disk 504 such as a hard drive (HDD) or portable storage medium or may be stored remotely.

Further, the claimed subject matter and/or claims are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the computing device communicates, such as a server or computer.

Further, in some embodiments, the claimed subject matter may be provided as, enabled by, or involve the use of a utility application, background daemon, or component of an operating system, or combination thereof, executing in conjunction with CPU 701, 703 and an operating system such as Microsoft Windows 7, Microsoft Windows 10, Microsoft Windows 11, UNIX, Solaris, LINUX, Apple MAC-OS and other systems known to those skilled in the art.

The hardware elements in order to achieve the computing device may be realized by various circuitry elements, known to those skilled in the art. For example, in some embodiments, CPU 501 or CPU 503 may be a Xenon or Core processor from Intel of America or an Opteron processor from AMD of America, or may be other processor types that would be recognized by one of ordinary skill in the art. In some embodiments, the CPU 501, 503 may be implemented on an FPGA, ASIC, PLD or using discrete logic circuits, as one of ordinary skill in the art would recognize. In some embodiments, CPU 501, 503 may be implemented as multiple processors cooperatively working in parallel to perform the instructions of the inventive processes described above.

The exemplary computing device in FIG. 5 can also includes a network controller 506, such as an Intel Ethernet PRO network interface card from Intel Corporation of America, which may be useful for interfacing with network 560. As can be appreciated, the network 560 can be a public network, such as the Internet, or a private network such as an LAN or WAN network, or any combination thereof and can also include PSTN or ISDN sub-networks. The network 560 can also be wired, such as an Ethernet network, or can be wireless such as a cellular network including EDGE, 3G, 4G and 5G wireless cellular systems. The wireless network can also be Wi-Fi, Bluetooth, or any other wireless form of communication that is known.

In some embodiments, the computing device further includes a display controller 508, such as a NVIDIA Geforce GTX or Quadro graphics adaptor from NVIDIA Corporation of America, which may be useful for interfacing with a display 510, such as a Hewlett Packard HPL2445w LCD monitor. In some embodiments, a general purpose I/O interface is included which can 512 interface with a keyboard and/or mouse 514 as well as a touch screen panel 516 on or separate from display 510. In some embodiments, a general purpose I/O interface also connects to a variety of peripherals 518 including printers and scanners, such as an Office-Jet or Desk-Jet from Hewlett Packard. In some embodiments, a sound controller 520 is also provided in the computing device such as Sound Blaster X-Fi Titanium from Creative, to interface with speakers/microphone 522 thereby providing sounds and/or music.

The general purpose storage controller 524 can connect the storage medium disk 504 with communication bus 526, which may be an ISA, EISA, VESA, PCI, or similar, for interconnecting all of the components of the computing device. A description of the general features and functionality of the display 510, keyboard and/or mouse 514, as well as the display controller 508, storage controller 524, network controller 506, sound controller 520, and general purpose I/O interface 512 is omitted herein for brevity as these features are known.

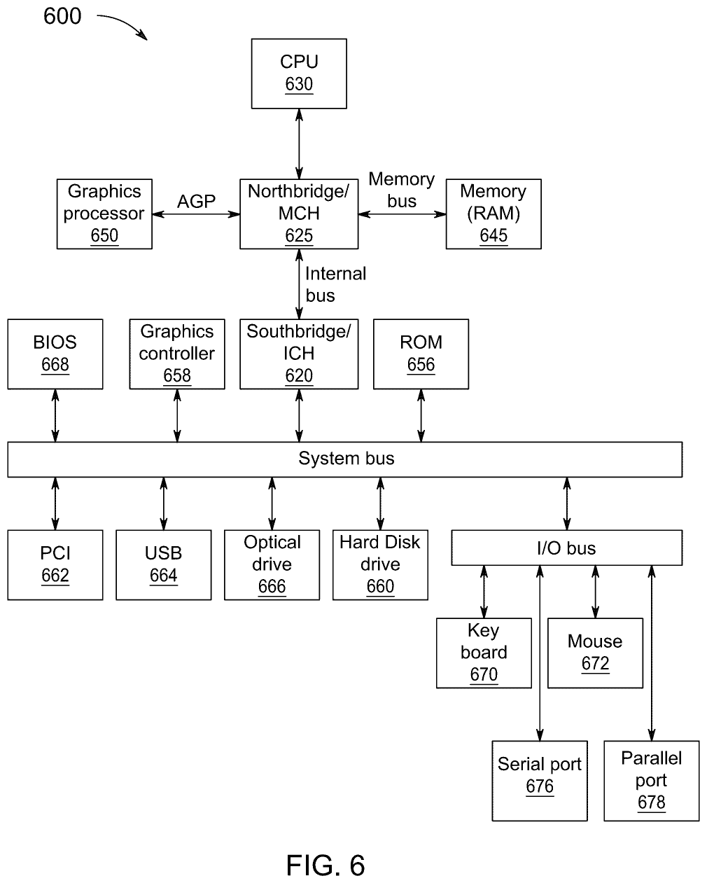

The exemplary circuit elements described in the context of the present disclosure may be replaced with other elements and structured differently than the examples provided herein. Moreover, circuitry configured to perform features described herein may be implemented in multiple circuit units (e.g., chips), or the features may be combined in circuitry on a single chipset, for example, as shown on FIG. 6.

FIG. 6 shows a schematic diagram of an exemplary data processing system, according to certain embodiments, for performing the functions of the exemplary embodiments. The data processing system is an example of a computer in which code or instructions implementing the processes of the illustrative embodiments may be located.

In FIG. 6, data processing system 600 employs a hub architecture including a north bridge and memory controller hub (NB/MCH) 625 and a south bridge and input/output (I/O) controller hub (SB/ICH) 620. The central processing unit (CPU) 630 is connected to NB/MCH 625. The NB/MCH 625 also connects to the memory 645 via a memory bus, and connects to the graphics processor 650 via an accelerated graphics port (AGP). The NB/MCH 625 can also connect to the SB/ICH 620 via an internal bus (e.g., a unified media interface or a direct media interface). The CPU Processing unit 630 may contain one or more processors and even may be implemented using one or more heterogeneous processor systems.

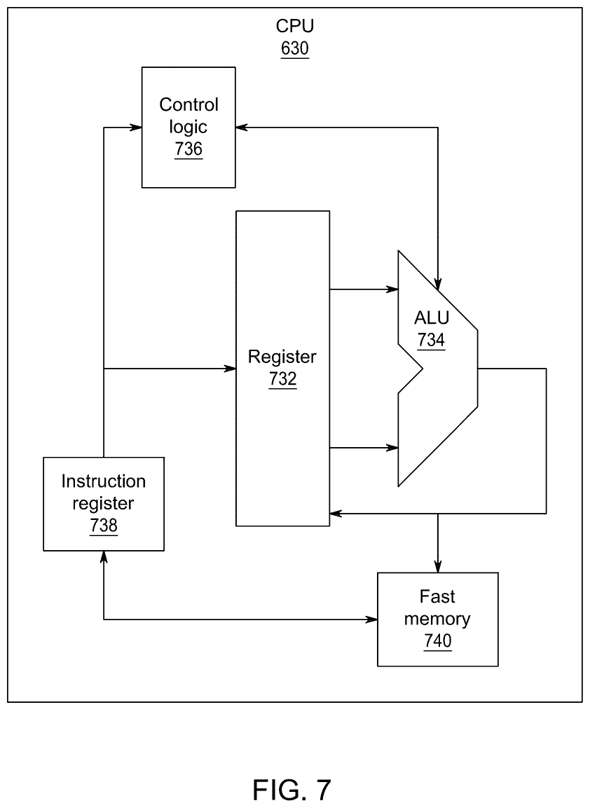

For example, FIG. 7 shows an exemplary implementation of CPU 630. In some implementations, the instruction register 738 retrieves instructions from the fast memory 740. At least part of these instructions can be fetched from the instruction register 738 by the control logic 736 and interpreted according to the instruction set architecture of the CPU 730. Part of the instructions can also be directed to the register 732. In some implementations, the instructions can be decoded according to a hardwired method. In some implementations the instructions can be decoded according to a microprogram that translates instructions into sets of CPU configuration signals that are applied sequentially over multiple clock pulses. After fetching and decoding the instructions, the instructions can be executed using the arithmetic logic unit (ALU) 734 that loads values from the register 732 and performs logical and mathematical operations on the loaded values according to the instructions. The results from these operations can be feedback into the register and/or stored in the fast memory 740. In some implementations, the instruction set architecture of the CPU 630 can use a reduced instruction set architecture, a complex instruction set architecture, a vector processor architecture, a very large instruction word architecture. Furthermore, the CPU 630 can be based on the Von Neuman model or the Harvard model. The CPU 630 can be, for example, a digital signal processor, an FPGA, an ASIC, a PLA, a PLD, or a CPLD. Further, the CPU 630 can be an x86 processor by Intel or by AMD; an ARM processor, a Power architecture processor by, e.g., IBM; a SPARC architecture processor by Sun Microsystems or by Oracle; or other known CPU architecture.

Referring again to FIG. 6, the data processing system 600 can include, for example, a SB/ICH 620 coupled through a system bus to any of an I/O Bus, a read only memory (ROM) 656, universal serial bus (USB) port 664, a flash binary input/output system (BIOS) 668, and a graphics controller 658. PCI/PCIe devices can also be coupled to SB/ICH 688 through a PCI bus 662.

The PCI devices may include, for example, Ethernet adapters, add-in cards, and PC cards for notebook computers. The Hard disk drive 660 and CD-ROM 666 can use, for example, an integrated drive electronics (IDE) or serial advanced technology attachment (SATA) interface. In one implementation the I/O bus can include a super I/O (SIO) device.

Further, the hard disk drive (HDD) 660 and optical drive 666 can also be coupled to the SB/ICH 620 through a system bus. In some embodiments, a keyboard 670, a mouse 672, a parallel port 678, and a serial port 676 can be connected to the system bus through the I/O bus. Other peripherals and devices that can be connected to the SB/ICH 620 using a mass storage controller such as SATA or PATA, an Ethernet port, an ISA bus, a LPC bridge, SM-Bus, a DMA controller, and an Audio Codec.

Moreover, the present disclosure is not limited to the specific circuit elements described herein, nor is the present disclosure limited to the specific sizing and classification of these elements. For example, the skilled artisan will appreciate that the circuitry described herein may be adapted based on changes on battery sizing and chemistry or based on the requirements of the intended back-up load to be powered.

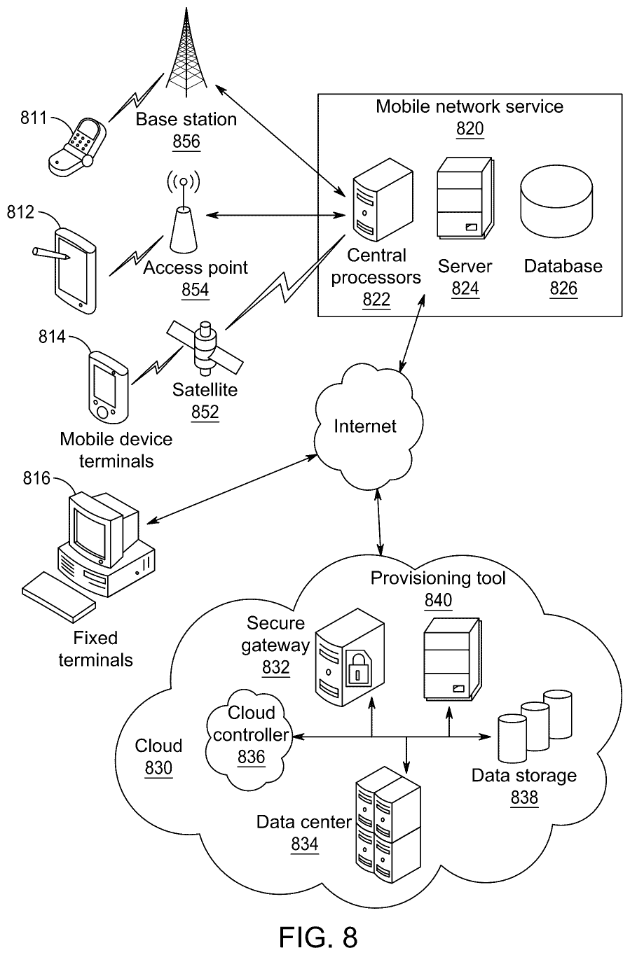

The functions and features described herein may also be executed by various distributed components of a system. For example, one or more processors may execute these system functions, wherein the processors are distributed across multiple components communicating in a network. The distributed components may include one or more client and server machines, such as cloud 830 including a cloud controller 836, a secure gateway 832, a data center 834, data storage 838 and a provisioning tool 840, and mobile network services 820 including central processors 822, a server 824 and a database 826, which may share processing, as shown by FIG. 8, in addition to various human interface and communication devices (e.g., display monitors 816, smart phones 810, tablets 812, personal digital assistants (PDAs) 814). The network may be a private network, such as a LAN, satellite 852 or WAN 854, or be a public network, may such as the Internet. Input to the system may be received via direct user input and received remotely either in real-time or as a batch process. Additionally, some implementations may be performed on modules or hardware not identical to those described. Accordingly, other implementations are within the scope that may be claimed.

The examples below are intended to further illustrate protocols for constructing and using the hand tremor treatment device and/or system and are not intended to limit the scope of the claims.

Where a numerical limit or range is stated herein, the endpoints are included. Also, all values and subranges within a numerical limit or range are specifically included as if explicitly written out.

### EXAMPLES

FIG. 1A shows a three-quarters view of an exemplary embodiment of a hand tremor treatment device, according to the present specification. The device is shown with the dorsal pad oriented toward the viewer. The exemplary device is shown with a touchscreen (off). The exemplary device shown in FIG. 1A may be a primary device as described above. The exemplary device shown in FIG. 1A may be configured for a pediatric (child) user.

FIG. 1B shows a three-quarters view of an exemplary embodiment of a hand tremor treatment device, according to the present specification. The device is shown with the dorsal pad oriented toward the viewer. The exemplary device does not include a touchscreen. The exemplary device shown in FIG. 1B may be a secondary device as described above. The exemplary device shown in FIG. 1A may be configured for a pediatric (child) user.

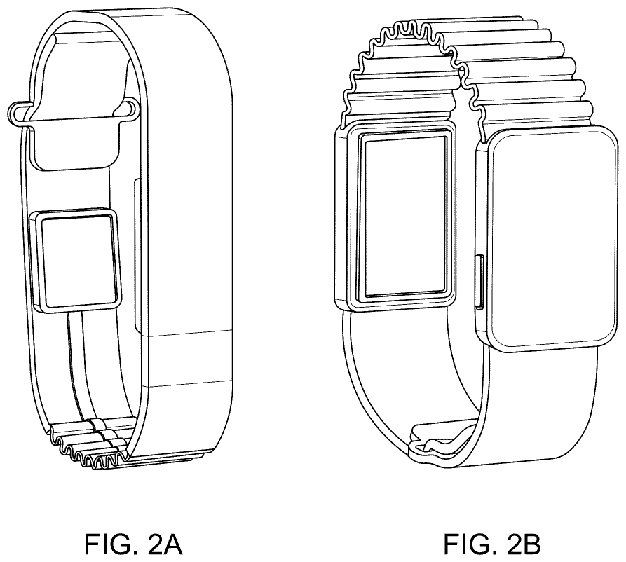

FIG. 2A shows a three-quarters view of an exemplary embodiment of a hand tremor treatment device, according to the present specification. The device is shown with the dorsal pad oriented toward the viewer. The exemplary device does not include a touchscreen. The exemplary device shown in FIG. 2A may be a secondary device as described above. The exemplary device shown in FIG. 2A may be configured for an adult user.

FIG. 2B shows a three-quarters view of an exemplary embodiment of a hand tremor treatment device, according to the present specification. The device is shown with the dorsal pad oriented toward the viewer. The exemplary device is shown with a touchscreen (off). The exemplary device shown in FIG. 2B may be a primary device as described above. The exemplary device shown in FIG. 2B may be configured for an adult user.

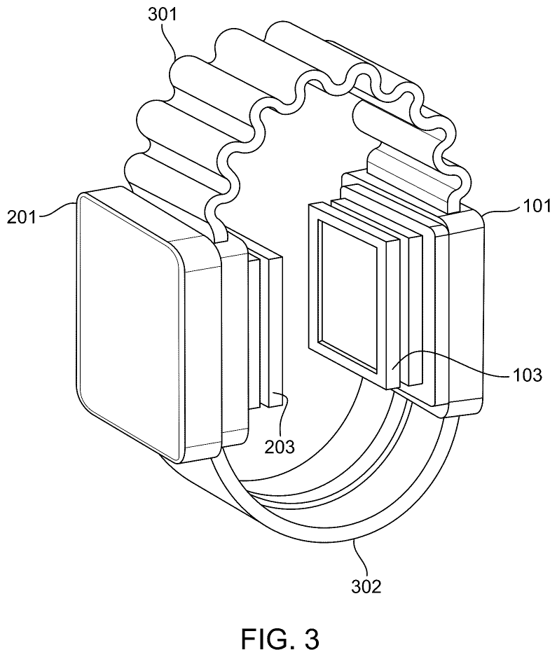

FIG. 3 shows an exemplary embodiment of a hand tremor treatment device, according to the present specification. The exemplary device includes a ventral pad 101 configured to be positioned on a ventral surface of a wrist of a subject and including a ventral compression-inducing device 103. The ventral vibration-inducing device is contained within the ventral pad 101. The exemplary device also includes a dorsal pad 201 configured to be positioned on a dorsal surface of a wrist of the subject and including a dorsal compression-inducing device 203. The dorsal vibration-inducing device is contained within the dorsal pad 201. The exemplary device also includes a lateral strap 301 connecting the ventral pad 101 and the dorsal pad 201 and configured to pass over a lateral surface of a wrist of the subject. The exemplary device also includes a medial strap 302 connecting the ventral pad 101 and the dorsal pad 201 and configured to pass over a medial surface of a wrist of the subject.

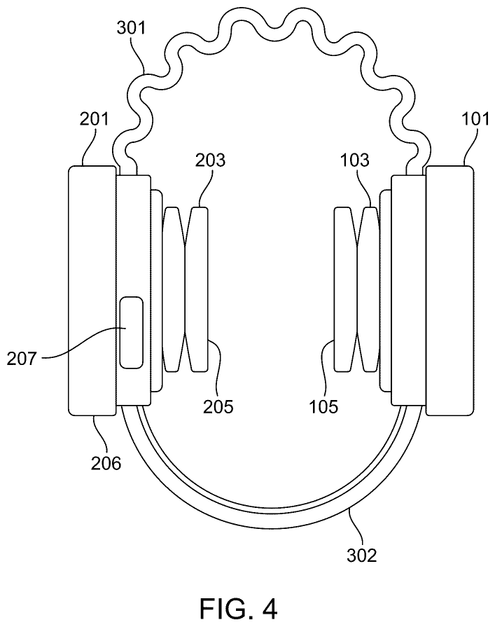

FIG. 4 shows an exemplary embodiment of a hand tremor treatment device, according to the present specification. The exemplary device includes a ventral pad 101 configured to be positioned on a ventral surface of a wrist of a subject and including a ventral compression-inducing device 103 (shown as an airbag in a partially or fully inflated state). The ventral vibration-inducing device is contained within the ventral pad 101. The ventral pad 101 also includes a ventral wrist contact plate 105. The exemplary device also includes a dorsal pad 201 configured to be positioned on a dorsal surface of a wrist of the subject and including a dorsal compression-inducing device 203 (shown as an airbag in a partially or fully inflated state). The dorsal pad 201 also includes a dorsal wrist contact plate 205. The dorsal pad 201 also includes a display 206 and a button 207 that is capable of functioning as a user input device. The dorsal vibration-inducing device is contained within the dorsal pad 201. The exemplary device also includes a lateral strap 301 connecting the ventral pad 101 and the dorsal pad 201 and configured to pass over a lateral surface of a wrist of the subject. The exemplary device also includes a medial strap 302 connecting the ventral pad 101 and the dorsal pad 201 and configured to pass over a medial surface of a wrist of the subject.

Numerous modifications and variations of the present invention are possible in light of the above teachings. It is therefore to be understood that, within the scope of the appended claims, the invention may be practiced otherwise than as specifically described herein.

1. A hand tremor treatment device, comprising
a ventral pad configured to be positioned on a ventral surface of a wrist of a subject and including a ventral vibration-inducing device and a ventral compression-inducing device;
a dorsal pad configured to be positioned on a dorsal surface of a wrist of the subject and including a dorsal vibration-inducing device and a dorsal compression-inducing device;
a lateral strap connecting the ventral pad and the dorsal pad and configured to pass over a lateral surface of a wrist of the subject;
a medial strap connecting the ventral pad and the dorsal pad and configured to pass over a medial surface of a wrist of the subject; and
a controller configured to detect a hand tremor onset and in response to detecting the hand tremor onset induce compression via at least one selected from the ventral compression-inducing device and the dorsal compression-inducing device and to induce vibration via at least one selected from the ventral vibration-inducing device and the dorsal vibration-inducing device.

2. The hand tremor treatment device of claim 1, wherein the lateral strap and the medial strap are flexible.

3. The hand tremor treatment device of claim 1, wherein at least one selected from the group consisting of the lateral strap and the medial strap is adjustable to securely conform to a wrist of a subject.

4. The hand tremor treatment device of claim 1, wherein ventral compression-inducing device and the dorsal compression-inducing device each include an inflatable airbag.

5. The hand tremor treatment device of claim 4, wherein the inflatable airbag includes a pump.

6. The hand tremor treatment device of claim 5, wherein the pump is configured to inflate and deflate the airbag in response to an airbag signal from the controller.

7. The hand tremor treatment device of claim 1, wherein the ventral vibration-inducing device and the dorsal vibration-inducing device are each selected from the group consisting of a rotary vibration motor and a piezoelectric linear vibration motor.

8. The hand tremor treatment device of claim 1, wherein the hand tremor treatment device comprises at least one vibration-inducing device configured to produce vibration in a first frequency range and at least one vibration-inducing device configured to produce vibration in a second frequency range.

9. The hand tremor treatment device of claim 8, wherein the first frequency range is 0.1 Hz to 1 kHz.

10. The hand tremor treatment device of claim 8, wherein the second frequency range is 10 kHz to 2.5 MHz.

11. The hand tremor treatment device of claim 1, further comprising an accelerometer, wherein the controller is configured to detect hand tremor onset using the accelerometer.

12. The hand tremor treatment device of claim 1, further comprising an angular velocity sensor, wherein the controller is configured to detect hand tremor onset using the angular velocity sensor.

13. The hand tremor treatment device of claim 1, wherein the ventral pad further comprises a user input device.

14. A hand tremor treatment system, comprising
a first hand tremor treatment device, comprising
a first ventral pad configured to be positioned on a ventral surface of a wrist of a subject and including a first ventral compression-inducing device and a first ventral vibration-inducing device;
a first dorsal pad configured to be positioned on a dorsal surface of a wrist of the subject and including a dorsal compression-inducing device and a first dorsal vibration-inducing device;
a first lateral strap connecting the first ventral pad and the first dorsal pad and configured to pass over a lateral surface of a wrist of the subject;
a first medial strap connecting the first ventral pad and the first dorsal pad and configured to pass over a medial surface of a wrist of the subject;
a first wireless communication device; and
a controller, and
a second hand tremor treatment device, comprising
a second ventral pad configured to be positioned on a ventral surface of a wrist of a subject and including a ventral compression-inducing device and a second ventral vibration-inducing device;
a second dorsal pad configured to be positioned on a dorsal surface of a wrist of the subject and including a dorsal compression-inducing device and a second dorsal vibration-inducing device;
a second lateral strap connecting the second ventral pad and the second dorsal pad and configured to pass over a lateral surface of a wrist of the subject;
a second medial strap connecting the second ventral pad and the second dorsal pad and configured to pass over a medial surface of a wrist of the subject; and
a second wireless communication device, wherein
the controller is configured to detect a hand tremor onset and in response to detecting the hand tremor onset induce compression via at least one selected from the first ventral compression-inducing device, the first dorsal compression-inducing device, the second ventral compression-inducing device, and the second dorsal compression-inducing device and to induce vibration via at least one selected from the first ventral vibration-inducing device, the first dorsal vibration-inducing device, the second ventral vibration-inducing device and the second dorsal vibration-inducing device; and
the first wireless communication device is configured to receive a signal from the controller and transmit the signal to the second wireless communication device.

15. The hand tremor treatment system of claim 14, wherein the first ventral pad further comprises a user input device.

16. A method of treating a hand tremor in a subject, the method comprising
detecting a hand tremor onset using the hand tremor treatment device of claim 1, and
inducing compression via at least one selected from the ventral compression-inducing device and the dorsal compression-inducing device and to induce vibration via at least one selected from the ventral vibration-inducing device and the dorsal vibration-inducing device.

17. The method of claim 16, further comprising
determining a hand tremor movement frequency; and
inducing vibration based on the hand tremor frequency.

18. The method of claim 16, wherein the controller comprises an angular velocity sensor and the controller is configured to detect hand tremor onset using the angular velocity sensor.

19. The method of claim 16, wherein the controller comprises an accelerometer and the controller is configured to detect hand tremor onset using the accelerometer.

20. The method of claim 16, further comprising
determining a hand tremor movement intensity; and
inducing vibration and compression based on the hand tremor intensity.
